(19) United States
(12) Patent Application Publication
(10) Pub. No.: US 2011/0319608 A1
(43) Pub. Date: Dec. 29, 2011

(54) PROCESS FOR PREPARING A MIXED SALT OF GLUCOSAMINE SULFATE AND AN ALKALI METAL CHLORIDE

(76) Inventors: Mahesh Devdhar, Mumbai (IN); Trupti Darge, Mumbai (IN); Rahul Jadhav, Mumbai (IN)

(21) Appl. No.: 12/602,621

(22) PCT Filed: Nov. 5, 2009
(86) PCT No.: PCT/EP09/07915

§ 371 Date: May 18, 2010

(51) Int. Cl. C07H 5/10

(52) U.S. Cl. 536/54

(57) ABSTRACT

The present invention relates to a process for preparing a mixed salt of glucosamine sulfate and an alkali metal chloride. The process comprises the consecutive steps of:
a) suspending glucosamine hydrochloride in a non-aqueous solvent,
b) adding thereto a solution of a basic alkali metal salt in water, and
c) adding thereto a solution of an alkali metal hydrogen sulfate in water
   whereby the mixed salt is obtained.

### CROSS-REFERENCE TO RELATED APPLICATIONS

This application is a 35 U.S.C. §371 national phase entry of PCT/EP2009/007915, filed Nov. 5, 2009, which is incorporated herein by reference.

### FIELD OF THE INVENTION

The present invention relates to a process for producing a mixed salt of glucosamine sulfate and an alkali metal chloride.

### BACKGROUND OF THE INVENTION

Glucosamine is a nutraceutical supplement that is known to provide relief in arthritis and joint pain. It is believed that glucosamine relieves inflammation in joint and helps in restoration of damaged cartilage. D-Glucosamine is used in the form of a salt such as glucosamine sulfate or glucosamine hydrochloride. The solid glucosamine sulfate is very hygroscopic and its amino group is readily oxidized in air. Formation of a mixed salt of glucosamine sulfate with an alkali metal chloride confers chemical stability to glucosamine sulfate and makes it less hygroscopic.

Processes for preparing a mixed salt of glucosamine sulfate and an alkali metal chloride have been described by several documents of the state of the art.

For example, U.S. Pat. No. 4,642,340 describes a process for preparing a mixed salt of glucosamine sulfate and sodium chloride by dissolving stoichiometric quantities of glucosamine sulfate and sodium chloride in water, followed by precipitation using a liquid precipitant which is miscible with water.

U.S. Pat. No. 5,847,107 describes a process in which stoichiometric quantities of glucosamine hydrochloride and a preselected sulfate are dissolved in a quantity of water variable between 4.5 to 6.5 times the weight of the sulfate. The resulting mixed glucosamine salt is precipitated by the addition of a liquid precipitating agent which is miscible with water.

Alternatively, U.S. Pat. No. 5,843,923 and U.S. Pat. No. 5,902,801 each describe a process in which water is removed by a freeze drying step. Thereby, glucosamine hydrochloride and a metal sulfate are contacted in water to form an aqueous solution of glucosamine sulfate metal chloride, which is thereafter freeze-dried at a temperature and at a reduced pressure for such period of time that at least about 90 wt % of the water is removed.

EP-A-0214642 relates to a process in which mixed salts are produced by dissolving a glucosamine base in water, adding a stoichiometric quantity of concentrated sulfuric acid to arrive at glucosamine sulfate and dissolving the stoichiometric quantity of alkaline or earth alkaline metal halide in the solution thus obtained. Precipitation is caused by adding a water-miscible organic solvent, such as isopropanol. Free glucosamine base is prepared by treating glucosamine hydrochloride with an ethanolic solution of a tertiary base such as triethyl amine.

U.S. Pat. No. 6,472,380 refers to a process in which glucosamine hydrochloride is contacted with a metal hydroxide in water to form a first aqueous solution of the free glucosamine base and a chloride of the metal. The first aqueous solution is then acidified with sulfuric acid to form a second aqueous solution of glucosamine sulfate and the chloride of the metal, said second aqueous solution being freeze-dried.

U.S. Pat. No. 6,812,223 relates to a process for the preparation of glucosamine sulfate metal salts having low metal content comprising the steps of

i) reacting glucosamine hydrochloride and a metal hydrogen sulfate selected from sodium hydrogen sulfate and potassium hydrogen sulfate in stoichiometric ratio in water;
ii) precipitating the resulting glucosamine sulfate metal salt in the presence of a water miscible organic solvent taken in a proportion of four to ten parts by volume with respect to the solution of step i); and
iii) filtering the reaction mass.

U.S. Pat. No. 7,435,812 describes a method carried out by

(a) placing glucosamine hydrochloride and a stoichiometric excess of sodium sulfate in water,
(b) heating the mixture obtained in the preceding step (a),
(c) cooling the mixture, and
(d) recovering the solid present in said cooled mixture by filtration.

According to the examples given in U.S. Pat. No. 7,435,812, the initial cycle yield is 74.1%. Recycling of the mother liquor with a calculated addition of glucosamine hydrochloride and sodium sulfate at a first and a second recycling step led to a combined yield of 85.4% and 89.6%, respectively.

In all documents of the state of the art cited above, water is used as a solvent for dissolving the reactants. Glucosamine sulfate metal chlorides mixed salts are highly soluble in water and to obtain good yields, either a precipitant is added, as for example according to U.S. Pat. No. 4,642,340, U.S. Pat. No. 5,847,107, EP-A-0214642 and U.S. Pat. No. 6,812,223, or a freeze-drying step is introduced, as for example according to U.S. Pat. No. 5,843,923, U.S. Pat. No. 5,902,801 and U.S. Pat. No. 6,472,380, whereby water is removed.

The use of a precipitant, on the one hand, has the disadvantage that it needs to be removed from the final product, which involves laborious purification steps. In addition, recycling of the precipitant is relatively complex and the precipitant is thus often discarded with the mother liquor. Both aspects have a negative impact on the efficiency of the process, in particular regarding large scale production.

The introduction of a freeze-drying step, on the other hand, is also relatively complex and requires—in particular for large scale production—sophisticated technical equipment, thus rendering the process very costly.

Additionally, the yields of the mixed salt of glucosamine sulfate and an alkali metal chloride obtained according to the process of the present invention are relatively poor.

### SUMMARY OF THE INVENTION

The object of the present invention is thus to provide an easy and cost-efficient process for the production of a mixed salt of glucosamine sulfate and an alkali metal chloride which leads to a highly pure product in yields superior to the ones obtained according to the state of the art.

The object is achieved by the process according to the invention. Preferred embodiments of the process are defined herein.

### DETAILED DESCRIPTION OF THE PREFERRED EMBODIMENTS

The present invention pertains to a novel process for preparing a mixed salt of glucosamine sulfate and an alkali metal chloride which is very easy and cost efficient and thus in particular usable for large scale industrial production.

According to the present invention, glucosamine hydrochloride is reacted in a non aqueous solvent, such as methanol or ethanol, with a basic alkali metal salt solution and an alkali metal hydrogen sulfate solution—in general in stoichiometric quantities—to obtain a mixed salt of glucosamine sulfate and an alkali metal chloride. This product is separable by filtration.

The process according to the present invention allows very high yields of more than 90% to be obtained.

Further, it avoids the use of toxic chemicals, such as triethyl amine, and also avoids the use of a water removal step, such as freeze drying steps, which in general are very costly.

According to the present invention, exposure of the glucosamine base to extreme pH conditions is avoided by adding a basic alkali metal salt prior to the addition of the alkali metal hydrogen sulfate solution. Thus, the formation of degradation products is vastly decreased.

According to a particular embodiment, an alkali metal acetate solution having a pH in the range of 8.5 to 9.5 is added, prior to the adding of the alkali metal hydrogen sulfate solution having a pH in the range of 0.25 to 0.35. By measuring the pH of the reaction mixture, it has been found that on complete addition of one half mole of the alkali metal acetate solution, the pH of the reaction mixture measured directly was in the range of 7.7 to 8.0 and the pH of the reaction mixture diluted in 6 parts of water was in the range of 5.8 to 6.0. Similarly, on complete addition of the alkali metal hydrogen sulfate solution the pH of the reaction mixture measured directly was in the range of 2.7 to 2.9 and the pH of the reaction mixture diluted in 6 parts of water was in the range of 3.1 to 3.3.

Alkali metal acetate, such as sodium acetate and potassium acetate, as well as alkali metal hydrogen sulfate, such as sodium hydrogen sulfate and potassium hydrogen sulfate, are highly soluble in water and the total quantity of water required for the dissolution constitutes only 0.5 to 0.7 parts by weight of water per part by weight of glucosamine hydrochloride taken.

The process of the present invention results in a highly pure mixed salt of glucosamine sulfate and an alkali metal chloride. Specifically, a purity of more than 98% can be achieved, as measured by a chloride assay as well as a glucosamine base assay. In this regard, the glucosamine base content was estimated by the colorimetric method as described by Elson and Morgan (Elson, L. A. and Morgan, W. T. J. Biochem. J. (1933), 27, 1824).

According to a preferred embodiment of the process of the present invention, step b and/or c) are carried out during stirring. Thus, the reaction mixture, to which the solution according to step b) and c), respectively, is added, is stirred during addition.

According to a preferred embodiment of the process of the present invention, the reaction temperature in step b) and c) is maintained at 0° to 5° C., leading to a particularly high yield of the mixed salt.

The mixed salt is preferably separated from the reaction mixture by filtration and washed with a washing solution comprising the non-aqueous solvent used in step a) and water. This separation and purification is very easy to perform and leads to a highly pure product.

According to a further preferred embodiment, the weight ratio between the non-aqueous solvent and water in the washing solution is about 3 to 1 to about 3 to 2, most preferably about 3 to 1.

It is further preferred that the washed mixed salt is further washed with an additional washing solution essentially consisting of the non-aqueous solvent. Thus, no further solvent is required which further contributes to the cost efficiency of the process.

The non-aqueous solvent is preferably an alcohol, most preferably either methanol or ethanol due to their availability, which is of particular relevance in large scale production.

It is further preferred that under step a) one part by weight of glucosamine hydrochloride is suspended in 2 to 3 parts by weight of the non-aqueous solvent.

According to a further preferred embodiment, the basic alkali metal salt is a sodium salt and/or a potassium salt. According to a particularly preferred embodiment, the basic alkali metal salt is sodium acetate and/or potassium acetate, due to the high yields of more than 90% and the low level of degradation products obtained. Other basic alkali metal salts include alkali metal carbonates, in particular sodium carbonate and/or potassium carbonate, and alkali metal hydroxides, in particular sodium hydroxide and/or potassium hydroxide.

Further, particularly high yields are obtained by setting the molar ratio of the basic alkali metal salt to glucosamine hydrochloride to less than about 1.2 to 2.0, more preferably to less than about 1.05 to 2.0, most preferably to about 1 to 2.

In the solution referred to in step b) the weight ratio of the basic alkali metal salt, in particular alkali metal acetate, to water preferably ranges from about 1 to 0.5 to about 1 to 1.5.

The alkali metal hydrogen sulfate is in general sodium hydrogen sulfate and/or potassium hydrogen sulfate. Thus, a mixed salt of glucosamine sulfate with sodium chloride and/or potassium chloride can be readily obtained without further reactions.

According to a further preferred embodiment, the molar ratio of alkali metal hydrogen sulfate to glucosamine hydrochloride is less than about 1.1 to 2.0, more preferably less than about 1.05 to 2.0, most preferably about 1 to 2.

It is further preferred that in the solution referred to in step c), the weight ratio of alkali metal hydrogen sulfate to water ranges from about 1 to 0.75 to about 1 to 1.5.

The following examples serve to more fully describe the above-described invention, as well as to set forth the best modes contemplated for carrying out various aspects of the invention. It is to be understood that these examples in no way serve to limit the true scope of this invention, but rather are presented for illustrative purposes only. It will be understood that all proportions are given in parts by weight, unless otherwise indicated.

### Example 1

In a reaction vessel, ethanol (178 g) and glucosamine hydrochloride (75 g) were charged and during stirring, the suspension was cooled to 0-5° C. Potassium acetate (17.06 g) was dissolved in 9 ml of water and the resulting clear solution was then slowly added to the reaction vessel over a period of 20-30 minutes. On complete addition of the potassium acetate solution, the direct reaction pH measured was 7.88 and the pH of the reaction mixture sample diluted in 6 parts water was 5.90.

Potassium hydrogen sulfate (23.68 g) was dissolved in 36 ml of water and the clear solution was then added slowly to the reaction vessel over a period of 30-40 minutes and stirring was continued for further 30 minutes, while the temperature of reaction was maintained at 0-5° C. At this stage, on direct measurement the pH of the reaction mixture was 2.8 and the pH of the reaction mixture sample diluted with 6 parts water was 3.2. The product was then separated by filtration. The wet cake of glucosamine sulfate potassium chloride was washed three times with a 75% ethanol in water solution followed by a final wash with ethanol. Washed solids were dried at 40-45° C. under vacuum. The yield of snow white glucosamine sulfate potassium chloride was 100.3 g (95.25%). The glucosamine base content by colorimetric estimation was 99.58% the chloride assay by HPLC was 100.70, and the specific optical rotation was 50.3 degree. The absorbance value (at 280 nm) and % transmittance value (at 450 nm) where 0.12 and 98.12%, respectively.

### Example 2

Example 1 was repeated using sodium acetate (14.06 g) dissolved in 21 ml of water and sodium hydrogen sulfate monohydrate (24.01 g) dissolved in 18 ml of water. The yield of snow white glucosamine sulfate sodium chloride was 95.4 g (95.6%). The glucosamine base content by colorimetric estimation was 99.8%, the chloride assay by HPLC was 100.59%, the specific optical rotation was 53.92 degree, and the pH of a 2% solution was 4.01. The absorbance value (at 280 nm) and % transmittance value (at 450 nm) where 0.06 and 98.38%, respectively.

### Example 3

Example 1 was repeated using methanol (178 g) as solvent. The yield of glucosamine sulfate potassium chloride was 97.1 g (92.21%). The glucosamine base content by colorimetric estimation was 98.63%, the chloride assay by HPLC was 101.75% and the specific optical rotation was 50.75 degree. The absorbance value (at 280 nm) and % transmittance value (at 450 nm) where 0.18 and 99.12%, respectively.

1. A process for preparing a mixed salt of glucosamine sulfate and an alkali metal chloride, wherein the process comprises the consecutive steps of:
a) suspending glucosamine hydrochloride in a non-aqueous solvent,
b) adding thereto a solution of a basic alkali metal salt in water, and
c) adding thereto a solution of an alkali metal hydrogen sulfate in water whereby the mixed salt is obtained.

2. The process according to claim 1, wherein one or both of step b) and c) are carried out during stirring.

3. The process according to claim 1, the reaction temperature in step b) and c) being maintained at 0° to 5° C.

4. The process according to claim 1, wherein the mixed salt is separated from the reaction mixture by filtration and washed with a washing solution comprising the non-aqueous solvent used in step a) and water.

5. The process according to claim 4, wherein the weight ratio between the non-aqueous solvent and water in the washing solution is from about 3 to 2 to about 3 to 1.

6. The process according to claim 4, wherein the washed mixed salt is further washed with an additional washing solution essentially consisting of the non-aqueous solvent.

7. The process according to claim 1, wherein the non-aqueous solvent is an alcohol.

8. The process according to claim 1, wherein under step a) one part by weight of glucosamine hydrochloride is suspended in 2 to 3 parts by weight of the non-aqueous solvent.

9. The process according to claim 1, wherein the basic alkali metal salt is one or both of a sodium salt and a potassium salt.

10. The process according to claim 9, wherein the basic alkali metal salt is one or both of sodium acetate and potassium acetate.

11. The process according to claim 1, wherein the molar ratio of the basic alkali metal salt to glucosamine hydrochloride is less than about 1.2 to 2.0.

12. The process according to claim 1, wherein in the solution referred to in step b) the weight ratio of the basic alkali metal salt to water ranges from about 1 to 0.5 to about 1 to 1.5.

13. The process according to claim 1, wherein the alkali metal hydrogen sulfate is one or both of sodium hydrogen sulfate and potassium hydrogen sulfate.

14. The process according to claim 1, wherein the molar ratio of alkali metal hydrogen sulfate to glucosamine hydrochloride is less than about 1.2 to 2.0.

15. The process according to claim 1, wherein in the solution referred to in step c), the weight ratio of alkali metal hydrogen sulfate to water ranges from about 1 to 0.75 to about 1 to 1.5.

16. The process according to claim 7, wherein the alcohol is selected from the group consisting of methanol, ethanol, and mixtures thereof.

17. The process according to claim 11, wherein the molar ratio of the basic alkali metal salt to glucosamine hydrochloride is less than about 1.05 to 2.0.

18. The process according to claim 11, wherein the molar ratio of the basic alkali metal salt to glucosamine hydrochloride is about 1 to 2.

19. The process according to claim 14, wherein the molar ratio of alkali metal hydrogen sulfate to glucosamine hydrochloride is less than about 1.05 to 2.0.

20. The process according to claim 14, wherein the molar ratio of alkali metal hydrogen sulfate to glucosamine hydrochloride is about 1 to 2.
